(19) United States
(12) Patent Application Publication
(10) Pub. No.: US 2006/0002994 A1
(43) Pub. Date: Jan. 5, 2006

(54) Responsive liposomes for ultrasonic drug delivery

(76) Inventors: James L. Thomas, Cedar Crest, NM (US); Hung-Yin Lin, Taipei (TW); Natalia Rapoport, Sandy, UT (US)

Correspondence Address: TRASK BRITT, P.O. BOX 2550, SALT LAKE CITY, UT, 84110, US

(21) Appl. No.: 11/087,142

(22) Filed: Mar. 23, 2005

Related U.S. Application Data
(60) Provisional application No. 60/555,911, filed on Mar. 23, 2004.

(51) Int. Cl. A61K 9/127

(52) U.S. Cl. 424/450

(57) ABSTRACT

This invention relates to biotechnology, more particularly, to an improved liposomal drug delivery system. Liposomes treated with or incorporating a surface active dopant, such as those containing polymers or oligomers of ethylene glycol as their hydrophilic “headgroup” component, have strongly increased permeabilizability when exposed to ultrasound. Permeabilizability was measured by the rate of release of self-quenching fluorescent dye at concentrations that caused no increase in permeability in the absence of ultrasound. The surface active dopants reached maximal effectiveness at about 1% of their critical micelle concentration. As disclosed by the present invention, surface active dopants, such as a PEG-lipid and a PLURONIC® triblock copolymer and a PEG-PBLA block copolymer, can be irreversibly incorporated into liposomes to give formulations for use as drug delivery vehicles.

### CROSS-REFERENCE TO RELATED APPLICATIONS

This application claims the benefit of the filing date of U.S. Provisional Patent Application Ser. No. 60/555,911, filed Mar. 23, 2004, for “Responsive Liposomes For Ultrasonic Drug Delivery,” the contents of which are incorporated herein by reference.

### TECHNICAL FIELD

This invention relates to biotechnology, more particularly, to an improved liposomal drug delivery system utilizing ultrasound and surface active dopants.

### BACKGROUND

Liposomes have long been thought to be promising drug delivery vehicles, owing to their biocompatibility and their ability to entrap and transport hydrophilic cargo. An important step in delivery is the release of an entrapped compound at the delivery site. If a vehicle is well targeted (either actively, or, for example, by bound antibodies [1-3] or passively through extravasation [4]), slow transmembrane permeation of the active drug may be acceptable. However, a triggered and rapid release of liposomal contents would be a useful improvement: lower targeting efficiency could be tolerated, and higher local drug doses would be readily achievable. In optimal cases, no targeting (other than localized triggering) would be necessary.

Ultrasound is an attractive method for triggering liposome release. Biomedical ultrasound is relatively non-invasive, yet penetrates through tissues and is able to be focused to an accuracy of millimeters. Moreover, high intensity ultrasound has long been used to break multilamellar liposomes into small unilamellar structures [5]. Thus, it is clearly capable of breaking liposomal membranes. Ultrasound appears to activate or enhance the pharmacological activity of some drugs; it can enhance drug transport through tissues and across cell membranes.

Ultrasound can also create a hyperthermic condition that can enhance the destruction of cancer cells. Due to the similarity in the material properties of cell membranes and liposome membranes, intense ultrasound can also destroy cells by disrupting cellular membranes. This cell membrane damage may be useful for treating malignancies [6] or in gene therapy [7, 8]. However, in other circumstances, it is desirable to have delivery vehicles that can be triggered with minimal damage to the surrounding tissues.

In U.S. Pat. No. 6,649,702 to Rapoport et al., the contents of which are incorporated by this reference, methods are disclosed in which a micelle is stabilized against degradation upon dilution. The micelle comprises molecules of a block polymer having a hydrophobic block and a hydrophilic block. The hydrophobic block forms a core of the micelle with corona from the hydrophilic block. The methods for stabilizing the core are (1) by chemically cross-linking, (2) incorporating a hydrophobic oil (vegetable oil) in the core to render it more hydrophobic and stable, and (3) incorporating a cross-linked interpenetrating network of a stimuli-responsive hydrogel into the core. The hydrogel is responsive to any stimuli, but preferably temperature or pH. A substance, for example, a drug, can be incorporated into the dense inner core of the micelles. When subjected to ultrasound, the micelles release the substance, and then reversibly revert to a stable dense core and re-encapsulate the substance when the ultrasound is turned off. By pulsing the ultrasound, one can controllably release the substance in a pulsed manner corresponding to the ultrasound signal.

### SUMMARY OF THE INVENTION

We have found that liposomes incorporating (or treated with) surface active dopants containing ethylene glycol polymers or oligomers show an enhanced response to ultrasound.

As described herein, ultrasound response was assayed by release of entrapped calcein, a self-quenching fluorescent dye. All the surface active molecules studied showed increasing effects with increasing concentration, but the sensitization saturated well below their critical micelle concentrations (CMCs). Surface active triblock copolymers (PLURONIC® Surfactants, BASF Aktiengesellschaft) showed temperature-dependent effects that were qualitatively consistent with temperature-dependent changes in their CMCs. Included among the molecules tested were two polyethylene glycol-lipids (PEG-lipids), which can be stably incorporated into liposomes when formed. PEG-lipids are used to retard the rapid immunological clearance of circulating liposomes in drug delivery formulations [9-13]; the application of ultrasound to stimulate release from these liposomes is therefore particularly attractive.

PEG-containing surfactants, including the PEG-lipids and PLURONIC® copolymers, enhance the permeabilizability of liposomes when exposed to ultrasound. The molecular origin of this effect is presently not understood. Not wishing to be bound by any particular theory, one theory is that these micelle-forming surfactants may stabilize membrane edges, and thus slow the resealing of ultrasound-induced holes in the liposomes. However, calculations show that, for 100 nm diameter liposomes, even small (about 1 nm) holes would result in the complete diffusional discharge of the liposome contents before resealing in a pure phospholipid membrane [26-28]. Membrane tensile strength, which is also weakened by these surfactants, does not seem to offer a clear explanation of the effect either: the saturation in ultrasound response occurs at surfactant concentrations where the membrane should be just beginning to weaken significantly, i.e., where the surfactant headgroups just begin to sterically compete. Moreover, in other experiments, the inclusion of cholesterol in phosphatidylcholine liposomes did not reduce their responsivity to ultrasound, even though cholesterol dramatically strengthens the membrane.

PLURONIC® polymeric surfactants, unlike the small-molecule surfactants, can be stably incorporated into (or inside) liposomes, and these liposomes can retain their responsivity to ultrasound when diluted. The combination of temperature and ultrasound responsivity of the PLURONIC®-containing liposomes acts to better target drug release, as shown in the Examples herein. In fact, therapeutic ultrasound results in local temperature increases, and highly temperature-sensitive liposomes have been designed with this stimulus in mind [29, 30].

### BRIEF DESCRIPTION OF THE DRAWINGS

FIG. 1. Leakage rate of Liposomes in insonation (o) or not (o) in PLURONIC® solution at 37° C.

FIG. 2. Leakage rate of cholesterol-containing liposomes in insonation (●) or not (∘) at 37° C.

FIG. 3. Leakage rate of PLURONIC® liposomes at different temperatures.

FIG. 4. Initial leakage rate of PEG-containing, surfactant-sensitized L-α-phosphatidyl choline (PC) liposomes. Pure PC liposomes, 0.06 μmoles, were added to different concentrations of TRITON® X-100 (Δ), TRITON® X-405 (▴), TWEEN® 20 (∘), and TWEEN® 80 (●) solutions. 1,2-dipalmitoyl-sn-glycero-3-phosphoethanolamine-N-methoxy(polyethylene glycol)2000 (DPPE-PEG2000, ▪) was incorporated into liposomes prior to extrusion. 20 kHz ultrasound at ca. 2 W/cm² was applied. Liposomes have maximum sensitivities to ultrasound in the presence of only 1-3% of the critical micelle concentration of these surfactants. In the absence of ultrasound, the leakage rate is <0.04 %/sec for all surfactants except TX-100.

FIG. 5. Leakage rates of liposomes incorporating PLURONIC® P-105 (A) or P85 (B) at different temperatures. PLURONIC® liposomes show a highly temperature-dependent response to ultrasound.

FIG. 6. Initial leakage rate as a function of PLURONIC® P-105 concentration at 20 μM (●), 2 μM (▴) and 0.2 μM (♦) liposomes (final lipid concentration) insonated with 20 kHz at 20% of full power and 25% duty cycle. Empty symbols are controls without insonation.

FIG. 7. Visualization of leakage on Petri dishes. 500 μL of 0.75 g/L BYPC/DPPE-PEG2000 (8 mol %) on 10 mL 0.4 wt % agarose gel in a 70 mm diameter Pyrex Petri dish. (Left plate) control, (middle plate) after applying 20% of maximum power with 25% duty cycle of 20 kHz ultrasound for 1 minute, (right plate) was taken with a camera adaptor which has a better throughput of the fluorescence emission.

FIG. 8. Florescence-Activated Cell Sorting (“FACS”) analysis of ovarian carcinoma A2780/ADR cells treated with or without PLURONIC® P-105.

FIG. 9. FACS analysis of ovarian carcinoma A2780 tumors from nu/nu mice treated or not treated with PLURONIC® P-105.

FIG. 10. FACS analysis of ovarian carcinoma A2780/ADR cells and A2780 tumors either (A) treated with PLURONIC® P-105 or not treated with PLURONIC® P-105 and either (B) not sonicated (no US) or sonicated (US).

### DETAILED DESCRIPTION OF THE INVENTION

It has been found, that certain chemical dopants, when added to liposomes useful in drug delivery, strongly increase the ultrasonic permeabilizability of liposomes. This phenomenon can be measured, for instance, by the rate of release of self-quenching fluorescent dye at dopant concentrations that cause no increase in permeability in the absence of ultrasound. The phenomenon can be harnessed to provide more efficient targeted drug delivery using liposomes either incorporating, or in the presence of dopants.

Proof of this concept can be obtained through study of the susceptibility of phosphatidyl choline liposomes to rupture by ultrasound. Herein, it is shown that liposomes treated with (or incorporating) a surface active dopant, containing, for example, polymers, copolymers or oligomers of ethylene glycol as their hydrophilic “headgroup” component, reach maximal effectiveness at about 1% of their critical micelle concentrations (“CMC”). Using the roughly inverse relationship between CMC and membrane partition coefficient, it was found that the maximally effective concentrations correspond approximately to the onset of headgroup contact among the surfactants in the membrane.

More particularly, two surfactants, a PEG-lipid and a PLURONIC® triblock copolymer, can be irreversibly incorporated into liposomes which give formulations useful as the aforementioned drug delivery vehicles. Furthermore, the PLURONIC® polymer offers the possibility of additional temperature responsivity, owing to its highly temperature dependent CMC.

The dopant may be administered in simultaneously with the liposomally-delivered agent (or agents) as a co-injection, administered through incorporating the dopant into the liposomes themselves, and the dopant may be administered separately, prior to or after, administration of the liposomes loaded with a pharmaceutically active compounds, or any combination thereof. Preferably, the dopant and the pharmaceutical agent (or agents) is (are) incorporated into the liposomes and dopant is administered prior to administration of the liposome (which may also have dopant incorporated therein). As will be appreciated by one of skill in the art, the amount of surface active dopant administered in the situation where the surfactant is administered separately to the subject or area, will vary depending on the identity and chemical formula of the dopant, the route of administration, its regulatory approval, and so forth. The highest dose of dopant administered will typically be such that it is not toxic to the subject, while the lowest dose will typically be that sufficient to enhance the efficacy of the ultrasound at causing release of the agent or agents and cellular uptake of the agent or agents (e.g. through enhanced disruption of loaded liposomes (liposomes encapsulating an agent or drug) and/or cellular membranes). Determination of the appropriate and effective dosage level of dopant for a particular dopant and for this purpose will be within the ability of one of ordinary skill in the art.

Micelles formed from the nonionic triblock polymer PLURONIC® surfactants have been found to enhance drug delivery and cellular uptake at the tumor site [31], especially in the presence of ultrasound. To investigate this effect, different compositions of liposomes were used as cell membrane mimics and were assayed for membrane disruption by release of an entrapped fluorescent dye. As disclosed herein, it is reported that addition of such dopants as PLURONIC® Surfactants, for example, dramatically enhance the ultrasound responsiveness of lipid bilayer membranes and cholesterol increases the membrane stability in the presence of ultrasound. As further disclosed here, to show effectiveness of this approach, a liposomally-loaded pharmaceutically active compound was delivered to an area on a subject and exposed to ultrasound.

The invention is further explained with the aid of the following illustrative Examples.

### EXAMPLE I

Preparation of Liposomes

LIPIDS AND CHEMICALS: Egg yolk PC was purchased from Avanti Polar Lipids (Birmingham, Ala.). PLURONIC® P-105 was a generous gift from BASF Aktiengesellschaft.

Stock lipids, and/or PLURONIC® P85, and P-105, were dried from chloroform solution under nitrogen and then under vacuum overnight. For permeability measurements, the lipids were vortex mixed and resuspended in a 50 mM calcein solution. The lipid mixture was then passed through two stacked polycarbonate filters (NUCLEPORE® 100 nm, Whatman Inc., Clifton, N.J.) nineteen times in a “mini-extruder” (Avanti Polar Lipids, Birmingham, Ala.) [14]. Unentrapped calcein was then removed by size exclusion chromatography (SEC) in a SEPHADEX® G50-packed (20-80 μm) 0.9×10 cm column (Aldrich), and eluted with an isotonic buffer (5 mM HEPES, 0.6% NaCl, 1 mM EDTA, pH 7.6).

### EXAMPLE II

PLURONIC® P-105-Enhanced PC Liposome Permeabilization

ULTRASOUND APPARATUS AND FLUORESCENCE MONITORING OF CALCEIN RELEASE FROM LIPOSOMES: The 20 kHz ultrasonic processor (Model VCI30PB, Sonics & Materials, Inc., Newtown, Conn.) was immersed into a polystyrene cuvette through a clearance hole in a TEFLON® cap, while the cuvette was held in the fluorimeter, an SLM Aminco 8000. Excitation and emission wavelengths were set at 488 nm and 520 nm, respectively. The probe was immersed approximately 1 cm into a 3 mL sample, initially containing only buffer and a magnetic stir bar. After adding 60 μL of liposome stock solution (ca. 0.06 pmole lipid), the fluorescence signal was allowed to stabilize for ca. 100 seconds. Then, the sample was sonicated for 5 minutes at 20% of full sonicator power and 25% duty cycle (approximately 2 W/cm²). At the conclusion of each experiment, the detergent TRITON® X-100 was added to rupture the liposomes completely and to achieve complete calcein release.

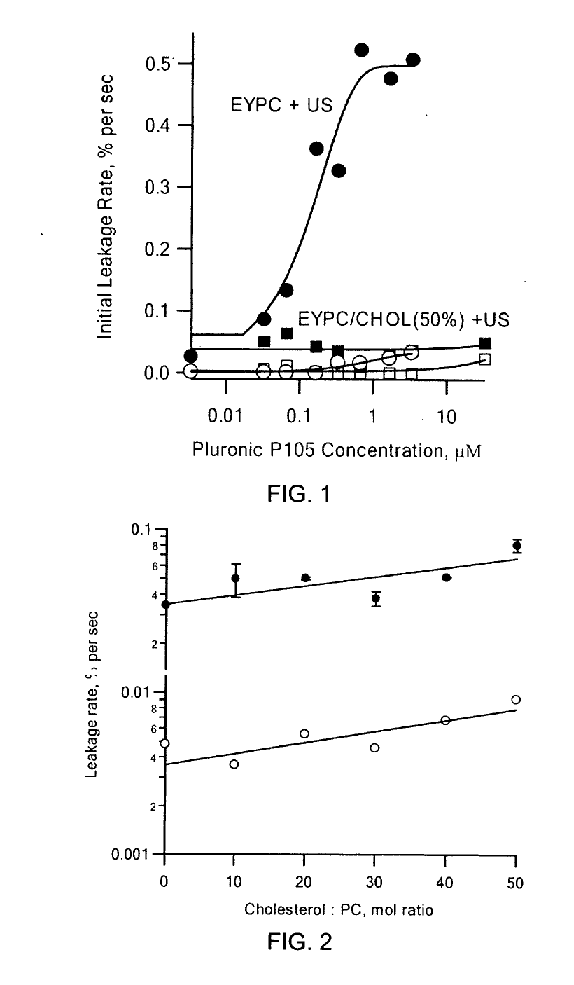

Comparisons of pure liposomes and phosphatidyl choline liposomes containing 50% cholesterol in the presence of various concentrations of PLURONIC® P-105 are shown in FIG. 1. The initial leakage rate of pure PC liposomes increases with increasing concentration of PLURONIC® P-105.

### EXAMPLE III

Effect of Cholesterol Concentration on Permeabilization

Liposomes were prepared according to Example I and analyzed as in Example II.

It is commonly known that cholesterol acts to make biological membranes more rigid such as those found in eukaryotes. The presence of cholesterol in the present system is therefore predicted to increase the rigidity of the liposomes; however, it was not known how this affects permeabilizability of the liposomes with dopant added. Thus, phosphatidyl choline (“PC”) liposomes containing different percentages of cholesterol were prepared and sonicated at 20% power (approximately 2 W/cm²) with the Sonics & Materials, Inc. probe sonicator. The results are depicted in FIG. 2. As shown in FIG. 2, insonation (●) or no insonation (∘), the initial leakage rates show a slight increase with increasing cholesterol concentration, indicating that inclusion of cholesterol in the liposomes does not prevent ultrasound-induced release. However, the initial leakage rate of pure PC liposomes was up to ten-fold higher than the liposomes with cholesterol added.

Thus, cholesterol-containing liposomes, such as those disclosed in the present invention, can be used with PLURONIC® P-105 for enhanced ultrasound-induced delivery. Thus, cholesterol may be utilized to increase liposome stability in the absence of ultrasound.

### EXAMPLE IV

Effect of Temperature on Permeabilization

Liposomes were prepared according to Example I and analyzed as in Example II.

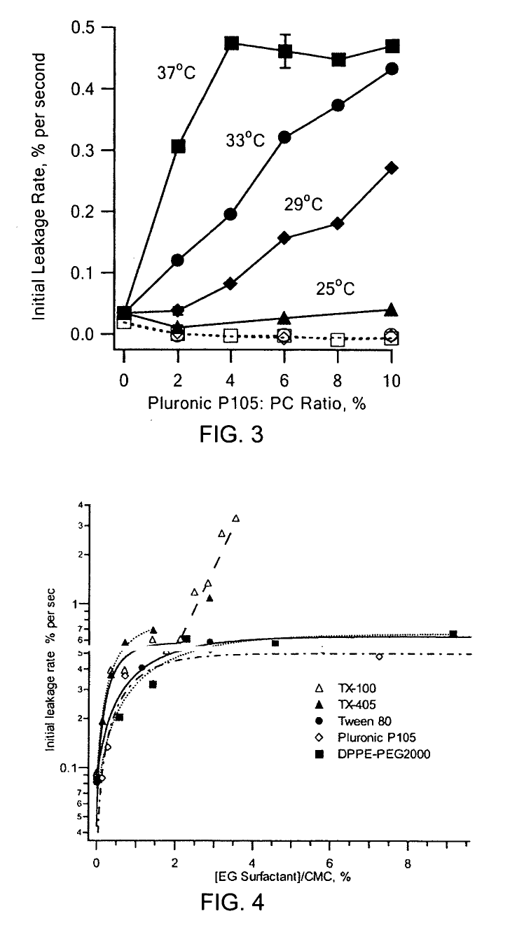

The CMC of PLURONIC® P-105 [25] is temperature dependent. PLURONIC® P-105 was incorporated into the liposomes during formation and extrusion. In FIG. 3, data is plotted as a function of temperature, where samples were tested at 25° C., 29° C., 33° C., and 37° C. (believed to mimic mammalian body temperature). The results show increasing sample temperature lowers the CMC of PLURONIC® P-105 and enhances the initial leakage rate.

Not wishing to be bound by any particular theory, one possible theory is that the temperature effect may indicate a change in the disposition of the polymer in the membrane as the hydrophobicity of the PPO block increases.

### EXAMPLE V

Effect of Addition of Dopant Before or After Liposome Extrusion

Liposomes were prepared according to Example I and analyzed as in Example II.

It was further observed that the same ratio of PLURONIC® to PC gives the same calcein leakage rate under insonation, regardless of whether PLURONIC® P-105 was added after or before liposome extrusion (not shown). Thus, it is possible that addition of PLURONIC® P-105 before extrusion does not yield polymer-liposome complexes that are significantly different from those formed by addition to pre-formed liposomes.

### EXAMPLE VI

PEG-Containing Surfactants Sensitize PC Liposomes to Ultrasound

MATERIALS: The lipids egg yolk PC, and 1,2-dipalmitoyl-sn-glycero-3-phosphoethanolamine-N-methoxy(polyethylene glycol)2000 (“DPPE-PEG2000”) were purchased from Avanti Polar Lipids (Birmingham, Ala.). PLURONIC® P-85 and P-105 are generous gifts from BASF Co. (Mount Olive, N.J.) as free samples. Calcein (fluorexon), and SEPHADEX® G50 were purchased from Aldrich Chemical Company (Milwaukee, Wis.). Agarose Type I-B: Low EEO, N[2-hydroxyethyl]piperazine-N′-[2-ethanesulfonic acid] (“HEPES”), and TRITON® X-100 (“TX-100”) were purchased from Sigma (St. Louis, Mo.). Sodium chloride and disodium ethylenediamine tetraacetate (“EDTA”), TRITON® X-405 (“TX-405”), TWEEN® 20 and TWEEN® 80 were purchased from Fisher Scientific (Fair Lawn, N.J.). Chemicals were used as received.

ULTRASOUND APPARATUS AND INSONATION: A 20 kHz ultrasonic processor (Model VC130PB, Sonics & Materials, Inc., Newtown, Conn.) was used [15, 16]. The probe of the sonicator was immersed into a polystyrene cuvette (1 mL path length) through a clearance hole in a TEFLON® cap, while the cuvette was held in the fluorimeter, an SLM Aminco 8000. The probe was immersed approximately 1 cm into a 3 mL sample, initially containing only buffer (5 mM HEPES buffer, 0.6% NaCl, 1 mM EDTA, pH 7.6) and a magnetic stir bar [17]. The ultrasonic processor was generally used at a 20% power setting; by measuring the rate of temperature increase in an insulated sample, the output at this setting was estimated to be approximately 2 W/cm². Low ultrasound intensities are unable to significantly permeabilize liposomes even in the presence of surface active agents [19].

Geometry and stirring rates remained constant throughout this Example. 60 μl (ca. 0.06 μmole lipid) of liposome stock solution was added through an injection port while stirring. The fluorimeter holds the cuvette in a brass heat transfer block. The temperature of the heat transfer block is controlled by a water circulator (model ISOTEMP® 3016, Fisher Scientific Co., Morris Plains, N.J.). Because ultrasonic energy causes substantial sample heating, we measured the temperature of the sample by direct immersion of a thermocouple probe (model HH2 1, Omega Engineering, Inc., Stamford, Conn.) and kept the sample temperature near 37° C. (or at the temperature indicated) (±1° C.) for all constant temperature experiments. The water circulator system was controlled using a PC RS232 serial port with LABVIEW® Software V5.0 (National Instruments Corp., Austin, Tex.). The duration and power of sonication was also computer controlled.

FLUORESCENCE MONITORING OF CALCEIN RELEASE FROM LIPOSOMES: Excitation and emission wavelengths were set at 488 nm and 520 nm, respectively. After adding 30 μl of liposome stock solution, the fluorescence signal (from residual unentrapped dye, and incomplete self-quenching of internal dye) was allowed to stabilize for ca. 100 seconds. Then, ultrasound was applied at the desired power, expressed as a percentage of full sonicator power. The duration of sonication was typically 5 minutes for each sample. At the conclusion of each experiment, the detergent TRITON® X-100 (60 μl of 1 wt %) was then added to rupture liposomes completely and to achieve complete calcein release. (The fluorimeter shutter was closed during TX-100 addition, resulting in the loss of signal at ca. 600 seconds.) The fluorescence increase was normalized as ΔJ(t)−[J(t)−Jₒ]/[Jₘₐₓ−Jₒ], where J is the measured fluorescence intensity, Jₒ the fluorescence intensity before liposome addition, and Jₘₐₓ the maximum fluorescence intensity after TRITON® X-100 addition [18].

In control experiments on buffer solutions, no direct signal from the insonation (i.e., sonoluminescence) was observed under our conditions. For experiments using the water-soluble surfactants, the surfactant was added to the cuvette prior to the addition of the liposomes. To find initial leakage rates, the early part of the release was fit to an exponential curve and the initial slope of the fit curve was normalized to the maximum total fluorescence increase (after liposome solubilization.)

RESULTS: Some ethylene glycol-containing surfactants can cause enhanced release of a self-quenching fluorescent marker dye from liposomes under 20 kHz ultrasound stimulation.[19] In FIG. 4, the rate of fluorescence increase caused by release of liposomally-entrapped calcein dye is shown in liposomes treated with five different surfactants. The total surfactant concentrations were normalized to the CMCs (Table 1, below). CMCs for TRITON® X-100, TRITON® X-405, PLURONIC® P-105 [20] and TWEEN® 80 were obtained from the literature [21]. All surfactants, except DPPE-PEG2000, were dissolved in the buffer prior to the introduction of the liposomes. DPPE-PEG2000 was incorporated into the liposomes prior to extrusion. For all surfactants except TRITON® X-100, the rate of fluorescence increase in the absence of ultrasound was <0.04% per second, i.e., off the bottom of the figure. (At the higher TRITON® X-100 concentrations, the spontaneous leakage was >0.11% per second.) All of the surfactants induced about a 10-15 fold increase in the response to 20 kHz ultrasound. More remarkably, the surfactants reached maximal effectiveness at concentrations of about 1% of the critical micelle concentration. The plateaus at these concentrations do not appear to be instrumental in creating transport artifacts—no significant increases in release occurred when stirring was made more rapid. Furthermore, the different surfactants plateau at different levels. (However, it is possible that there is some contribution from diffusive transport through a boundary layer near the sonicator tip; but at this relatively low ultrasonic frequency, high intensities will be found only near the tip itself.)

TABLE 1
Critical micelle concentrations (CMC) and estimated partition
coefficients (K) of several detergents assayed. For membrane-
solubilizing detergents, the product of the CMC and the partition
coefficient is of order unity [22].

| Detergent | CMC (μM) | K (J/M) |
|---|---|---|
| TX-100 | 240 | 4167 |
| TX-405 | 810 | 1235 |
| TWEEN ® 20 | 80 | 12500 |
| TWEEN ® 80 | 100 | 10000 |
| DPPE-PEG2000 | 70 | 14286 |
| PLURONIC ® P-105 @ 37° C. | 23 | 43478 |

Critical micelle concentrations (CMC) and estimated partition coefficients (K) of several detergents assayed. For membrane-solubilizing detergents, the product of the CMC and the partition coefficient is of order unity [22].

To a first approximation, the partitioning of a detergent into the lipid membrane should vary inversely with its CMC [22]; thus, equal surfactant to CMC ratios in FIG. 4 should roughly correspond to equal membrane concentrations. There appear to be only small differences (ca. a factor of 3) among the saturation concentrations of the detergents studied. For the PEG-lipid, the saturation concentration in ultrasound responsivity corresponds to the transition between “mushroom” and “brush” packing regimes for the PEG headgroup.[23, 24] It thus seems likely that saturation effects observed with the other surfactants are also related to the onset of headgroup packing strain.

This result indicates that the membrane composition, more than the membrane mechanics, is important for the ultrasound response.

### EXAMPLE VII

Effect of Entrapped or Transmembrane PLURONIC® Surfactants on Liposomes

Methods used in this Example are as described in the previous Examples unless otherwise noted.

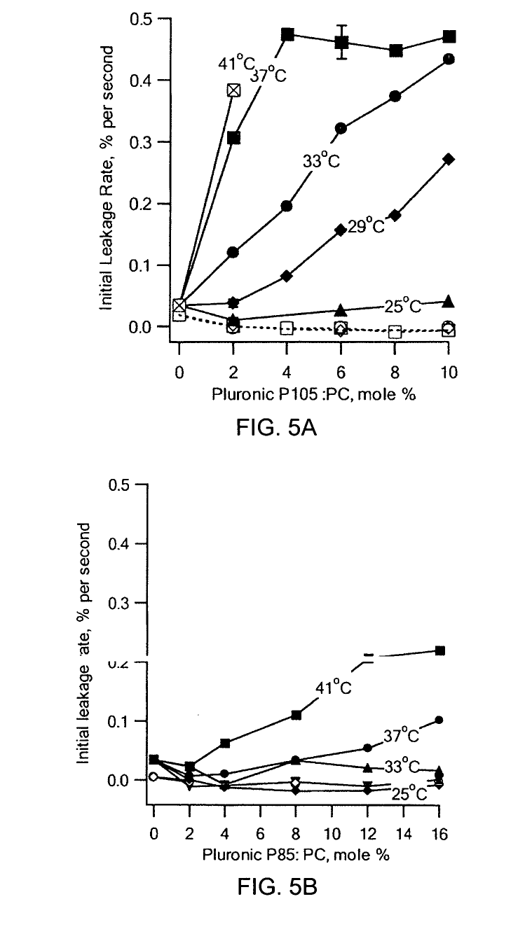

Like PEG-lipids, PLURONIC® triblock polymers have a very low CMC, and thus might be stably incorporated into liposomal delivery vehicles. To promote an “integral” association between PLURONIC® P-105 and the liposomes, the PLURONIC® was added to the lipid prior to hydration and extrusion. (Unfortunately, both PLURONIC® and liposomes run in the void volume of the SEPHADEX® G50 column; some unbound PLURONIC® may still be present.) PLURONIC® Surfactants incorporated in this fashion increase PC liposome responsivity to ultrasound, as shown in FIG. 5(A).

Of particular interest is the temperature dependence of the response: at 25° C., there is almost no sensitization at a mole ratio of 0.1, while at 37° C., a mole ratio of 0.04 causes a 10-fold increase in responsivity. The changes in responsivity are correlated with the increasing hydrophobicity (and thus decreasing critical micelle concentration) of P-105 with increasing temperature [25]. The smaller P-85 also showed a temperature-dependent response, but was generally much less effective. (See, FIG. 5(B)).

### EXAMPLE VIII

Effect of Dilution on Responsivity of Liposomes

Methods used in this Example are as described in the previous Examples unless otherwise noted.

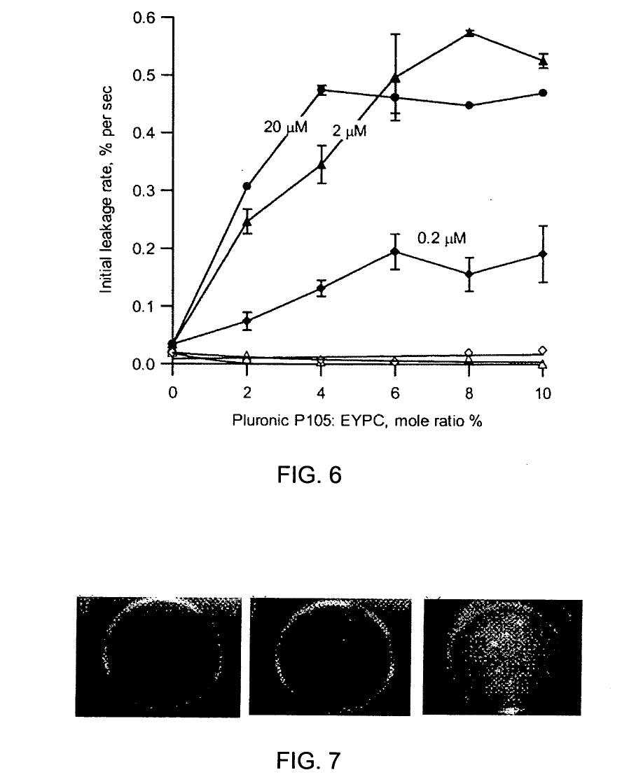

Incorporation of PLURONIC® P-105 into liposomes preserves their ultrasound responsivity upon dilution, as shown in FIG. 6. The liposomes used in FIG. 5 were diluted 10-fold and 100-fold with buffer, and their response to 20 kHz ultrasound was measured. Successive dilutions is believed to promote the dissociation of superficially absorbed PLURONIC®, while PLURONIC® that is either entrapped within the liposomes or bound in a transmembrane conformation should be retained. Using the rough estimate for the partition coefficient given in Table 1, a 100-fold dilution (to a lipid concentration of 0.2 μM) should remove essentially all of the superficial P-105. However, these liposomes are still able to release their contents at a rate >5-fold higher than controls with no PLURONIC® P-105.

### EXAMPLE IX

Visualization of Effect of Ultrasound

Methods used in this Example are as described in the previous Examples unless otherwise noted.

VISUALIZATION OF LOCALIZED RELEASE: 10 mL of agarose 0.4 wt % in buffer (5 mM HEPES buffer, 0.6% NaCl, 1 mM EDTA, pH 7.6) was added to a Pyrex Petri dish (70×50, No. 3140) at about 70° C., then 500 -L of EYPC/PEG-DPPE 2000 (8 mol %) liposome stock solution (ca. 0.5 pmole lipid) was added on the top of agarose gel layer at about 35° C. The 20 kHz ultrasonic processor (model VC13OPB, Sonics & Materials, Inc., Newtown, Conn.) was used. The probe of the sonicator was immersed into the agarose layer (1 mm in depth) directly. The agarose layer was about 5 millimeters deep.

Duration of 20 kHz ultrasound applied to the sample was 1 min. at 20% of full power and with 25% duty cycle, at each spot where release was desired. Before and after localized release, the sample was viewed in a UV hood (SPECTROLINE® model CC-80, Spectronics Corporation, Westbury, N.Y.) and was excited by 365 nm long wave ultraviolet light (SPECTROLINE® model ENF-280C, Spectronics Corporation, Westbury, N.Y.). Images were taken in a darkened room by a digital camera (model PowerShot G2, Canon U.S.A., Inc., Lake Success, N.Y.) was controlled using a personal computer USB port using Canon RemoteCapture V2.1 software. The software was adjusted with ACDSEE® 5.0 (ACD Systems Ltd., Arlington, Tex.) with the following parameters: black (1), white (238) and gamma (1.0).

RESULTS: The ability of ultrasound to cause localized release of liposomally entrapped calcein dye is visually demonstrated in the Example shown in FIG. 7. In this Example, 5 mL of 66 mM PC/DPPE-PEG2000 (8 mol %) liposomes were layered onto a 0.4 wt % agarose gel and allowed to permeate the gel for 12 hours. The gel was imaged using a UV illuminator before (left plate) and after (middle plate) applying ultrasound at five distinct spots. FIG. 7, middle plate, shows clear fluorescence at the insonated spots, resulting from the relief of the calcein self-quenching. FIG. 7, right plate, shows the same sample, but taken through a camera adapter that transmits the fluorescent light better. FIG. 7 (right plate), taken 2 minutes after FIG. 7 (middle plate), shows some evidence of dye diffusion in the broadening of the fluorescent spots.

This measurement shows that entrapped or transmembrane PLURONIC® Surfactants can be used to effectively enhance ultrasonic delivery.

### EXAMPLE X

Effect of Priming Ovarian Cancer Cells with PLURONIC®

Methods used are as described in the previous Examples unless otherwise noted.

As further evidence supporting the results and conclusions of the preceding in vitro experiments, the effect of first introducing PLURONIC® to cells and solid, multi-drug resistant (MDR) tumors prior to administration of the target drug was analyzed. Doxorubicin, a common antitumor antibiotic, was first encapsulated into the liposomes as described in the prior Examples. Then, ovarian carcinoma A2780/ADR cells were exposed to PLURONIC® for 30 minutes then exposed to encapsulated doxorubicin for 30 minutes. Then the cells were sonicated at between 1-3 MHz, preferably 1 MHz, at 33% duty cycle corresponding to about 3.4 W/cm² power intensity.

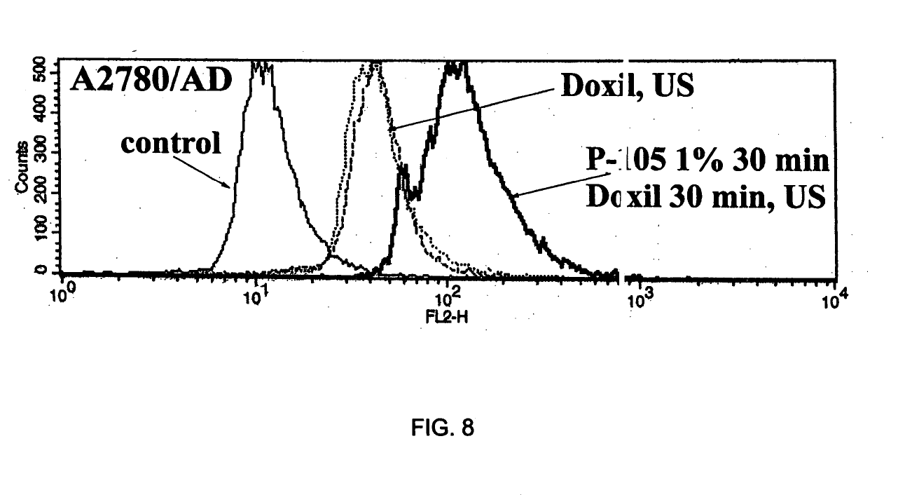

Results were obtained utilizing fluorescence microscopy and by labeling liposomes with calcein, as before. (See, FIG. 8). FIG. 8 shows a fluorescence-activated cell sorting (“FACS”) data display of cells treated with PLURONIC® (Doxil, US) and cells not treated with PLURONIC® P-105 (P-105 1% 30 min, Doxil 30 min, US). The data show a clear shift in the number of cell nuclei infiltrated by casein, and hence doxorubicin, upon treatment with PLURONIC® P-105 (dark-lined peak) verses those cells not treated with PLURONIC® P-105 (lighter-lined peak) and upon sonication.

### EXAMPLE XI

Effect of Priming Ovarian MDR Tumors with PLURONIC®

A similar experiment, as that described in the prior Example, Example X, was performed on ovarian carcinoma A2780 tumors in nu/nu mice. A 1% solution of PLURONIC® P-105 was first injected intravenously into the mice 1 hour prior to doxorubicin injection. Doxorubicin was physically loaded into micelles prepared from poly(ethylene glycol)-poly(beta-benzyl-L-aspartate) block copolymer (PEG-PBLA) and then injected at about 1-20 mg/kg, preferably at 6 mg/kg, either intravenously or intratumorally. Four to eight hours, preferably four hours, post-injection of doxorubicin, tumors were sonicated at between 1-3 MHz, preferably 1 MHz, ultrasound at 33% duty cycle corresponding to about 3.4 W/cm² power intensity.

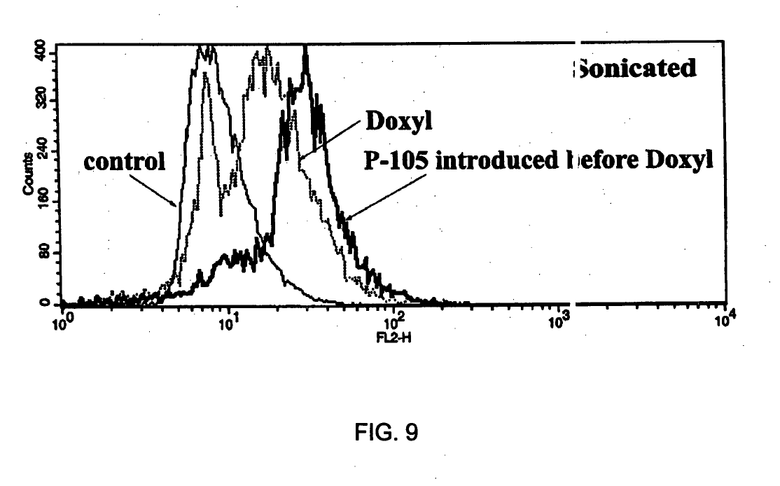

Results were obtained utilizing fluorescence microscopy and by labeling liposomes with calcein, as before. (See, FIG. 9). FIG. 9 clearly shows that PLURONIC® P-105-treated tumor cells (dark-lined peak) incorporated much more doxorubicin than PLURONIC® P-105 non-treated (lighter-lined peak) tumor cells.

The results of this study show that local ultrasonic irradiation of solid tumors in vivo results in a substantially increased drug accumulation in the tumor cells without significant effect on the drug or drug carrier uptake by the cells of other organs. This further indicates that a substantial degree of drug targeting to only the tumor cell is possible using this technique.

### EXAMPLE XII

Effect of Sonication on Efficient Infiltration of Target Cells with Doxorubicin

Methods used are as described in the previous Examples unless otherwise noted.

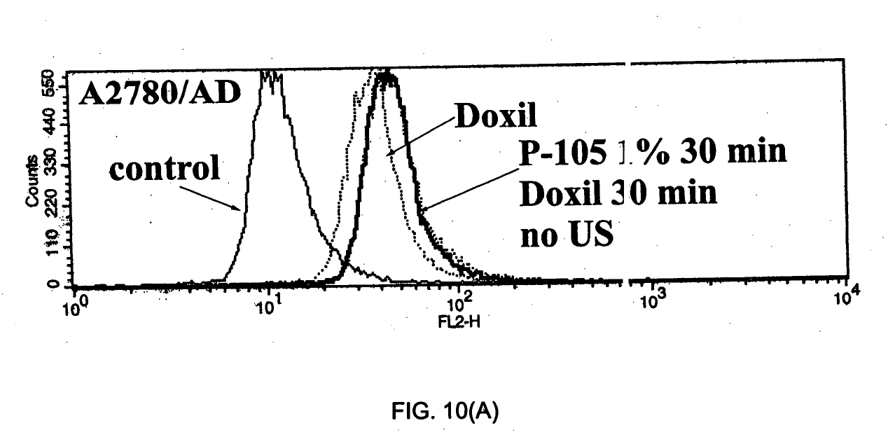

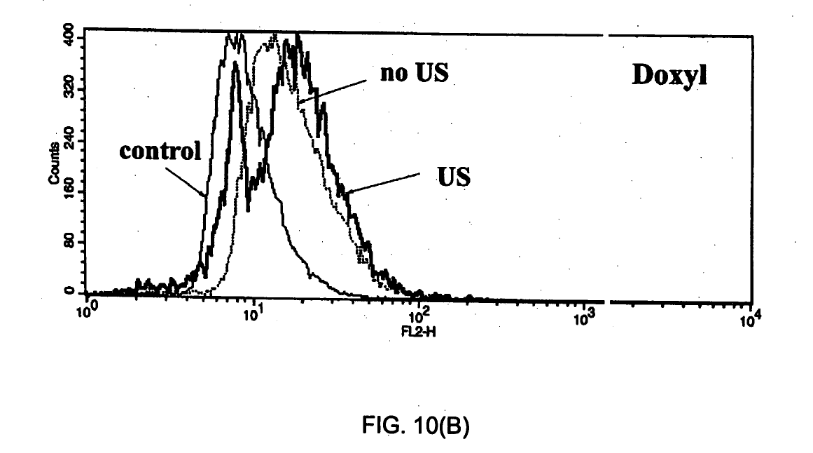

To determine what effect sonication had on targeting of tumor cells with PEG-PBLA-encapsulated doxorubicin, experiments similar to those disclosed in prior Examples were carried out either with sonication or without sonication. (See, FIG. 10). In FIG. 10(A), it can be seen that PLURONIC® P-105-treated A2780/ADR cells take up more doxorubicin (dark-lined peak) than the same cells not treated with PLURONIC® P-105 (lighter-lined peak). Furthermore, in comparing FIG. 10(A) to FIG. 8, one observes that sonication improves infiltration of PEG-PBLA-encapsulated doxorubicin when sonication is applied as described above.

Similar results are obtained in examining treated A2780 tumors in nu/nu mice. Nu/nu mice were treated as described above in Example XI except that in one sample sonication was not applied (light-lined peak in FIG. 10(B)). As shown in FIG. 10(B), sonication does improve cellular uptake of doxorubicin (darker-lined peak). Furthermore, when comparing FIG. 10(B) to FIG. 9, one observes that treatment with PLURONIC® P-105 dramatically increases uptake of doxorubicin.

Cumulatively, these experiments show that the following procedures incrementally increase uptake of doxorubicin to the target tumor cells: sonication, sonication in the presence of PEG-PBLA-encapsulated doxorubicin, pretreatment with PLURONIC® P-105 followed by sonication in the presence of PEG-PBLA-encapsulated doxorubicin.

All references, including publications, patents, and patent applications, cited herein are hereby incorporated by reference to the same extent as if each reference were individually and specifically indicated to be incorporated by reference and were set forth in its entirety herein. The following list of references are hereby incorporated by reference:
1. T. M. Allen, A. K. Agrawal, I. Ahmad, C. B. Hansen, and S. Zalipsky, I Liposome Res., 4, 1 (1994).
2. T. M. Allen and E. H. Moase, Adv. Drug Delivery Rev., 21, 117 (1996).
3. S. M. Moghimi, A. C. Hunter, and J. C. Murray, Pharmacol Rev., 53, 283 (2001).
4. C. Tordeux and J.-B. Fournier, Europhys. Lett., 60, 875 (2002).
5. C. Huang, Biochemistry, 8, 344 (1969).
6. M. W. Miller, Ultrasound in Medicine & Biology, 26, S59 (2000).
7. S. Bao, B. D. Thrall, R. A. Gies, and D. L. Miller, Cancer Res., 58, 219 (1998).
8. S. Bao, B. D. Thrall, and D. L. Miller, Ultrasound in Medicine & Biology, 23, 953 (1997).
9. T. M. Allen, Adv. Drug Delivery Rev., 13, 285 (1994).
10. T. M. Allen and A. Chonn, FEBS Letters, 223, 42 (1987).
11. T. M. Allen, C. Hansen, F. Martin, C. Redemann, and A. F. Yau-Young, Biochim. Biophys Acta, 1066, 29 (1991).
12. T. M. Allen, C. B. Flansen, and D. E. Lopes de Menezes, Adv. Drug Delivery Rev., 16, 267 (1995).
13. T. M. Allen, C. Hansen, and J. Rutledge, Biochim. BiophysActa, 981, 27 (1989).
14. M. J. Hope, M. B. Bally, G. Webb, and P. R. Cullis, Biochim. Biophys Acta, 812, 55 (1985).
15. G. A. Husseini, G. D. Myrup, W. G. Pitt, D. A. Christensen, and N. Y. Rapoport, J. Control. Release, 69, 43 (2000).
16. G. A. Husseini, N. Y. Rapoport, D. A. Christensen, J. D. Pruitt, and W. G. Pitt, Co/bids and Surfaces B: Biointerfaces, 24, 253 (2002).
17. M. W. Miller, D. L. Miller, and A. A. Brayman, Ultrasound in Medicine & Biology, 22, 1131 (1996).
18. N. Duzgunes, R. M. Straubinger, P. A. Baldwin, and a. et, Biochemistry, 24, 3091 (1985).
19. H.-Y. Lin and J. L. Thomas, Langmuir, 19, 1098 (2003).
20. M. Johnsson, M. Silvander, G. Karlsson, and K. Edwards, Langmuir, 15, 6314 (1999).
21. A. McPherson, Crystallization of Biological Macromolecules, Cold Spring Harbor Laboratory Press, Laguna Niguel, 1998.
22. H. Heerklotz and J. Seelig, Biophys. J., 78, 2435 (2000).
23. A. K. Kenworthy, S. A. Simon, and T. J. McIntosh, Biophys. 1, 68, 1903 (1995).
24. A. K. Kenworthy, K. Hristova, D. Needham, and T. J. McIntosh, Biophysical Journal, 68, 1921 (1995).
25. P. Alexandridis and T. A. Hatton, Colloids and Surfaces A: Physicochemical and Engineering Aspects, 96, 1 (1995).
26. E. Evans and W. Rawicz, Phys. Rev. Lett., 64, 2094 (1990).
27. D. V. Zhelev and D. Needham, Biochim. Biophys Acta, 1147, 89 (1993).
28. J. Moroz and P. Nelson, Biophys. J., 72, 2211 (b997).
29. J.-C. Leroux, E. Roux, D. Le Garrec, K. Hong, and D. C. Drummond, J. Control. Release, 72, 71 (2001).
30. G. R. Anyarambhatla and D. Needham, I. Liposome Res., 9, 491 (1999).
31. N. Rapoport, “Stabilization and activation of PLURONIC® micelles for tumor-targeted drug delivery,” Colloids and Surfaces B: Biointerfaces, vol. 16, pp. 93-111, 1999.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the scope of the appended claims.

What is claimed is:

1. A method of delivering an agent to an area associated with a subject, comprising:
incorporating said agent into a liposome, to create a loaded liposome;
administering a surface active dopant and said loaded liposome to the subject so as to allow the surface active dopant and loaded liposome to reach said area; and
subjecting the area to ultrasound, thus rupturing said liposome and thus delivering said agent to the area.

2. The method according to claim 1, wherein the liposome is a phosphatidyl choline liposome.

3. The method according to claim 1, wherein the loaded liposome is treated with, or has had incorporated therein, a composition comprising a surface active dopant.

4. The method according to claim 1, wherein the surface active dopant is a polymer, copolymer or oligomer having a hydrophilic moiety comprising ethylene glycol.

5. The method according to claim 3, wherein the surface active dopant is present in an amount of from about 0.01% to about 3% of the dopant's critical micelle concentration.

6. The method according to claim 1, wherein the surface active dopant is selected from the group consisting of a polyethylene glycol, L-α-phosphatidyl choline, beta-benzyl-L-aspartate, a triblock copolymer, a PLURONIC® surfactant, cholesterol, and mixtures thereof.

7. The method according to claim 1, wherein the ultrasound has an energy frequency of about 20 kHz.

8. The method according to claim 1, wherein the loaded liposome is heated at the area to promote rupturing of the liposome.

9. The method according to claim 1, wherein the area comprises a tumor.

10. The method according to claim 1, further comprising administering the dopant to the area before administering the loaded liposomes to the area.

11. The method according to claim 9, wherein an energy frequency of the ultrasound is about 1-3 MHz.

12. The method according to claim 1, wherein the agent has anticarcinogenic activity designed to treat cancer cells.

13. The method according to claim 12, wherein the agent is doxorubicin.

14. A liposome, wherein the improvement comprises:
incorporating into said liposome a surface active dopant so as to enhance the liposome's ability to rupture in the presence of ultrasound.

15. The liposome of claim 15, wherein the surface active dopant is present in an amount of from about 1% to about 3% of the dopant's critical micelle concentration.

16. The linosome of claim 15, wherein the surface active dopant is selected from the group consisting of a polyethylene glycol, L-α-phosphatidyl choline, beta-benzyl-L-aspartate, a triblock copolymer, a PLURONIC® surfactant, cholesterol, and mixtures thereof.

17. A method of treating an area associated with a subject with a medicament, said method comprising:
incorporating a medicament into a liposome, forming a loaded liposome and wherein said loaded liposome further comprises at least one dopant;
administering to a subject a surface active dopant;
waiting for an effective period of time;
administering to said subject said loaded liposome; and
applying ultrasound to the subject, thus rupturing said liposome and delivering said medicament to the subject.

18. The method of claim 18, wherein said medicament is useful in treating cancer.

19. A composition comprising:
a medicament, wherein said medicament is encapsulated in a liposome, and wherein said liposome is comprised of a lipid and a dopant selected from the group consisting of a polyethylene glycol, L-α-phosphatidyl choline, beta-benzyl-L-aspartate, a triblock copolymer, a PLURONIC® surfactant, cholesterol, and mixtures thereof.

20. A method of delivering an agent to an area associated with a subject, said method comprising:
incorporating a therapeutic amount of the agent into the liposomes of claim 15, to create a loaded liposome;
administering the loaded liposome to the subject;
applying ultrasound locally of an energy frequency of about 1 to about 20 MHz to the area rupturing the loaded liposome and delivering the agent to the area.
